(19) United States
(12) Patent Application Publication
(10) Pub. No.: US 2026/0021275 A1
(43) Pub. Date: Jan. 22, 2026

(54) CATHETER WITH POLYMER COATING OF VARIED FLEXIBILITY

(71) Applicant: Transit Scientific, LLC, Salt Lake City, UT (US)

(72) Inventors: Greg Method, Santa Monica, CA (US); Ryan Merriner, Salt Lake City, UT (US); Jennifer Arnold, Salt Lake City, UT (US)

(21) Appl. No.: 18/775,469

(22) Filed: Jul. 17, 2024

(51) Int. Cl. A61M 25/00

(52) U.S. Cl. CPC A61M 25/0054; A61M 25/0013; A61M 25/0045; A61M 25/008; A61M 2205/0266

(57) ABSTRACT

A catheter that is at least partially defined from a hypotube includes a polymer coating of varied flexibility on an outer surface of the hypotube. The polymer coating may include a plurality of adjacent sections that are continuous with each other, but have different flexibilities from each other. The sections may have different hardnesses, or durometers that impart them with their different flexibilities. Methods for manufacturing such catheters are also disclosed.

### TECHNICAL FIELD

This disclosure relates generally to catheters and, more specifically, to catheters that are at least partially defined from hypotubes. Even more specifically, this disclosure relates to a catheter that includes a polymer coating of varied flexibility on an outer surface of the hypotube. Methods for manufacturing catheters are also disclosed.

### RELATED ART

Standard angiography catheters are typically manufactured from thermoplastic materials by extrusion processes. Angiography pressures with a power injector can reach about 1,200 psi (about 8.275×10³ kpa), which may exceed the pressure ratings of many standard angiography catheters, which are around 800 psi (about (about 5.5×10³ kpa). Thus, the use of standard angiography catheters can be risky, as excessive pressures may cause such catheters to fail and, thus, may injure a subject on whom the angiography procedure is being conducted.

Microcatheters typically have thin walls and small diameters to enable them to navigate tiny veins and other vessels within a subject's body. Microcatheters that are used in cardiovascular applications may have outer diameters (ODs) of less than 6 French (F) (2 mm). Neurovascular microcatheters may have outer diameters as small as 2.3 F (0.77 mm). Because of their thin walls and small outer diameters, pushability, trackability, and torqueability requirements have limited the lengths of microcatheters. Typically, the upper limit on the length of a microcatheter is about 150 cm, with some microcatheters—particularly those with smaller outer diameters—being much shorter. In addition, to provide desired levels of pushability, trackability, and torqueability, microcatheters are typically tapered; stated another, way, the wall thickness and/or outer diameter is typically not uniform along the length of the microcatheter.

Hypodermic tubes, or “hypotubes,” have been used for a variety of purposes, including reinforcing portions of conventional thermoplastic catheters. A hypotube is a thin-walled tube formed from a metal or a metal alloy, such as a stainless steel, a nickel titanium alloy (e.g., nitinol, which stands for Nickel Titanium Naval Ordinance Laboratory; etc.), or the like. Hypotubes are useful for a variety of purposes. In the medical device industry, hypotubes have been manufactured in a variety of lengths and with outer diameters ranging from about 0.120 inch (11 gauge) to 0.005 inch (36 gauge). Among other purposes, hypotubes have been used to facilitate the introduction of catheters through a patient's anatomy.

While stainless steel hypotubes may enhance the ability of a catheter to glide (e.g., push, track, and torque) through a subject's anatomy, they are known for their tendency to kink, particularly when forced through tortuous paths. Nitinol has also been used to form hypotubes. While nitinol hypotubes are kink resistant, they are much more expensive to manufacture (particularly in developmental stages and in small quantities) than stainless steel hypotubes, and still lack sufficient flexibility to enable them to advance through many of the bends that are present in a subject's vasculature.

### SUMMARY

A catheter of this disclosure includes a hypotube and a polymer coating on an outer surface of the hypotube. The polymer coating has a varied flexibility along a length of the catheter. Stated another way, one portion of the polymer coating may have a different flexibility than one or more other portions of the polymer coating. In specific embodiments, a proximal portion of the polymer coating may be less flexible, or stiffer, than a distal portion of the polymer coating, which is more flexible. Optionally, the catheter may include a liner within a lumen of the hypotube. In embodiments where the catheter includes a liner, the polymer coating may be bonded to the liner.

The hypotube may be formed from any suitable material. For example, the hypotube may be made from a metal or a metal alloy. One example of a metal is stainless steel (e.g., 304 stainless steel, 316 stainless steel, etc.). An example of a metal alloy is a so-called “shape memory alloy,” such as a nickel titanium alloy (e.g., nitinol (nickel titanium Naval Ordinance Laboratory), etc.). As another example, the hypotube may be made from a polymer. Regardless of the material from which the hypotube is formed, it includes a wall that defines the inner surface and the outer surface of the hypotube, as well as lumen that extends through at least a portion of a length of the hypotube.

The hypotube may include one or more cuts or other features that enhance its flexibility. Flexibility enhancing cuts may extend partially around the hypotube in a manner that increases a flexibility of the hypotube without enabling elongation of the hypotube. Flexibility enhancing cuts may be oriented helically around the hypotube (i.e., at an non-perpendicular angle to a longitudinal axis of the hypotube when the hypotube is straight) and/or circumferentially around the hypotube (i.e., at a perpendicular angle to the longitudinal axis of the hypotube when the hypotube is straight). The flexibility enhancing cuts may have a configuration that varies the flexibility of the hypotube along its length. For example, the lengths of the flexibility enhancing cuts may be varied at different locations along the length of the hypotube (e.g., shorter flexibility enhancing cuts at more proximal locations along the length of the hypotube may render such locations stiffer or less flexible than more distal locations of the hypotube, which may include longer flexibility enhancing cuts; etc.). As another example, spacing between flexibility enhancing cuts may be varied along the length of the hypotube (e.g., flexibility enhancing cuts at more proximal locations along the length of the hypotube may be spaced apart from each other to render such locations stiffer or less flexible than more distal locations of the hypotube, where flexibility enhancing cuts may be positioned more closely to each other; etc.).

The polymer coating on the outer surface of the hypotube may include a plurality of sections of different flexibilities along its length. Adjacent sections may be continuous with each other, or their ends may be continuous and optionally comprise a transition between the adjacent sections.

Each section of the polymer coating or, more specifically, a flexibility of that section of the polymer coating may impart a corresponding portion of the length of the catheter with a particular flexibility, which may affect the pushability, trackability, and torqueability of that portion of the length of the catheter. The different flexibilities of the different sections of the polymer coating may result from different hardnesses, or different durometers, of the different sections, different thicknesses of the different sections, or some combination of hardness and thickness. For example, the polymer coating may include a plurality of sections with hardnesses that differ from each other while having substantially the same thickness. As another example, the polymer coating may include a plurality of sections that have the same hardness, but different thicknesses. As yet another example, a section of the polymer coating may have a hardness that differs from a hardness of at least one other section of the polymer coating and a thickness that differs from at least one other section (but not necessarily the same other section) of the polymer coating. In embodiments where the polymer coating includes sections with different thicknesses, a thinner hypotube may be used to minimize the maximum outer diameter of the catheter.

The polymer coating may extend into and through flexibility enhancing cuts and any other cuts or openings through the wall of the hypotube. The polymer coating may comprise a thermoplastic elastomer (TPE) that may flow into the flexibility enhancing cuts or other openings when heated to a sufficient temperature (e.g., a reflow temperature, etc.) and/or subjected to sufficient pressure. Polyether block amide (PEBA) (e.g., that available as PEBAX® polymer from Arkema SA of Colombes, France; that available as VESTAMID® E from Evonik Industries AG of Essen, Germany, etc.) is an example of a polymer from which the polymer coating may be formed. Polyamide 12, or nylon 12 (e.g., that available as VESTAMID® Care ML21 polymer from Evonik Industries AG, etc.) is another example of a polymer from which the polymer coating may be formed. Optionally, the polymer coating may bond to the hypotube.

The optional liner may be positioned within the lumen of the hypotube, adjacent to or against the inner surface of the wall of the hypotube, or adjacent to or against the inner surface of the hypotube. An inner surface of the liner may enhance the ability of fluids, embolics, etc., to flow through the lumen of the catheter. Such enhanced fluid flow may be achieved with a liner that has an inner surface that is smoother than, or has a lower coefficient of friction than, the inner surface of the hypotube. Without limitation, the liner may comprise polytetrafluoroethylene (PTFE).

In embodiments where the catheter includes an optional liner, the polymer coating may bond to the liner at locations where the polymer coating or a material of the polymer coating contacts the liner. For example, portions of the polymer coating or its material that extend through the flexibility enhancing cuts or other cuts or openings through the wall of the hypotube, portions of the polymer layer that extend beyond a distal and/or proximal end of the hypotube, etc., may contact and optionally bond to the liner.

In a more specific embodiment, the hypotube may comprise a stainless steel, the polymer coating may comprise PEBA or polyamide 12, and the optional liner may comprise PTFE. The polymer coating includes a plurality of regions (e.g., two regions, three regions, etc.) arranged in sequence and continuous with each other, with each region of the polymer coating having a hardness that differs from a hardness of each adjacent region of the polymer coating. For example, a polymer coating may include a proximal region over a proximal portion of the hypotube, an intermediate region over an intermediate region of the hypotube, and a distal region over a distal region of the hypotube, with a hardness of the proximal region being greater than a hardness of the intermediate region and the hardness of the intermediate region being greater than a hardness of the distal region.

A method for manufacturing a catheter may include cutting a hypotube to define cuts, including flexibility enhancing cuts, or other openings through a wall of the hypotube. A liner may be optionally introduced into a lumen of the hypotube. A plurality of polymer layers of different flexibilities (e.g., hardnesses, thicknesses, etc.) may be introduced over an outer surface of the hypotube, with the polymer layers arranged sequentially along the length of the hypotube and an end of each polymer layer contacting or superimposed with an end of each adjacent polymer layer. A heat shrink may be provided over the polymer layer. Once the polymer layers and heat shrink are in place over the outer surface of the hypotube, they may be heated.

Cutting the hypotube may comprise defining cuts or other openings in the hypotube or, more specifically, through a wall of the hypotube. Defining the cuts may comprise defining flexibility-enhancing cuts through the hypotube. Defining the flexibility enhancing cuts may comprise defining helically arranged flexibility enhancing cuts (i.e., cuts that are at a non-perpendicular angle to a longitudinal axis of the hypotube when the hypotube is straight). Alternatively or additionally, defining the flexibility enhancing cuts may comprise defining circumferentially arranged flexibility enhancing cuts (i.e., cuts that are at a perpendicular angle to the longitudinal axis of the hypotube when the hypotube is straight). The hypotube may comprise a metal (e.g., a stainless steel, a shape memory alloy, etc.), a polymer, etc. Cutting the hypotube may comprise laser cutting the hypotube.

Introducing the optional liner into the lumen of the hypotube may comprise introducing a tubular liner into the lumen of the hypotube. The liner may be formed from a material that is smoother, or has a lower coefficient of friction, than the inner surface of the hypotube (e.g., PTFE, etc.). The liner may be carried over a mandrel, and the mandrel may be used to introduce the liner into the lumen of the hypotube. Optionally, the mandrel may ensure that an outer surface of the liner remains in place adjacent to or against the inner surface of the hypotube as subsequent processes are conducted (e.g., while heating the polymer layer and the heat shrink, while removing the heat shrink, etc.).

Introducing the polymer layers over the outer surface of the hypotube may comprise introducing polymer layers in tubular form over the outer surface of the hypotube in an arrangement, or a sequence, that impart a finished polymer layer with desired variations in flexibility. The polymer layers may be positioned end-to-end along the length of the hypotube, with ends of adjacent polymer layers positioned to enable the formation of a continuous polymer layer (e.g., in close proximity to each other, in contact with each other, partially overlapping, or superimposed with, each other, etc.). Each polymer layer may be formed from a polymer that may flow when subjected to appropriate conditions (e.g., heat, pressure, etc.). The polymer of the polymer layer may bond to the optional liner when subjected to appropriate conditions (e.g., heat, pressure, etc.). Optionally, the polymer of each polymer layer may bond to the hypotube when subjected to appropriate conditions (e.g., heat, pressure, etc.). The polymer of each polymer layer may comprise a TPE (e.g., a PEBA, a polyamide 12, etc.).

Providing the heat shrink may include providing a tubular heat shrink over the polymer layers. The heat shrink may be placed over the polymer layers after the polymer layer have been introduced over and arranged on the hypotube. The heat shrink may shrink to a predetermined inner diameter, which may equal a desired outer diameter for the polymer layers and, thus, the catheter. The heat shrink may be formed from any suitable material (e.g., a polyolefin, such as polyethylene terephthalate (PET) (i.e., polyester), etc.).

The heat shrink and the polymer layers may be heated to a temperature sufficient to cause the polymer(s) of the polymer layers to flow (or reflow) (e.g., a reflow temperature of the polymer, a melting point of the polymer, etc.) and to cause the heat shrink to shrink. As the heat shrink is heated, its inner diameter shrinks. As the polymer layers are heated to such a temperature, the polymer(s) may flow. The polymer(s) of adjacent polymer layers may flow or reflow into each other and mix, which may provide a transition between the adjacent polymer layers and form a continuous polymer layer of varied flexibility from the adjacent polymer layers. Shrinkage of the heat shrink may facilitate flow of the polymer(s) into and through cuts or other openings through the hypotube. As the polymer(s) flows into and through cuts or other openings in the hypotube, the polymer(s) may contact the liner. In embodiments where a mandrel is present within the liner, the mandrel may ensure that the liner remains in contact with the inner surface of the hypotube and that an inner diameter of the liner and, thus, the catheter are maintained during as the polymer layers are heated and their polymer reflows.

At locations where the heated polymer(s) contact(s) the liner, the polymer(s) and, thus, the polymer layers, may bond to the liner. Such bonding may occur at cuts or other openings through the hypotube (i.e., locations through which the polymer has flowed). Optionally, such bonding may occur at locations where the liner and at least one polymer layer extend beyond an end of the hypotube (e.g., a distal end of the hypotube, etc.); together, these overlapping, bonded portions of the liner and polymer layer may define a tip of the catheter.

As the heat shrink is heated, it may also mold the polymer of the polymer layers, forcing the polymer layers against the outer surface of the hypotube. As portions of the polymer layers that contact surfaces of the hypotube (e.g., the outer surface of the hypotube, one or both ends of the hypotube, surfaces of cuts or other openings through the hypotube, etc.) are heated, those portions of the polymer layers may bond to the surfaces of the hypotube.

Once the heat shrink and the continuous polymer layer of varied flexibility cool, the heat shrink may be removed (e.g., cut, pulled, etc.) from the continuous polymer layer of the catheter. In embodiments where a mandrel remained in place during heating of the polymer layer and the heat shrink, the mandrel may removed (e.g., pulled, etc.) from the lumen of the liner and, thus, of the catheter.

Other aspects of the disclosed subject matter, as well as features and advantages of various aspects of the disclosed subject matter should be apparent to those of ordinary skill in the art from the foregoing, the ensuing description, the accompanying drawings, and the appended claims.

### BRIEF DESCRIPTION OF THE DRAWINGS

In the drawings:

FIG. 1 is a cross-sectional representation taken along a length of a catheter that includes a hypotube, an optional liner within a lumen of the hypotube, and a continuous polymer layer of varied flexibility over an outer surface of the hypotube, with the continuous polymer layer bonded to the liner;

FIG. 2 shows the hypotube of the catheter, as well as cuts formed in the hypotube;

FIGS. 2A, 2B, and 2C are cross-sectional representations of a distal portion, an intermediate portion, and a proximal portion of the catheter shown in FIG. 1;

FIG. 2D schematically illustrates a portion of another embodiment of a hypotube of a catheter, as well as cuts formed in the hypotube;

FIG. 3 is an enlarged cross-sectional representation of an intermediate portion of the catheter of FIG. 1, showing bonding of the continuous polymer layer to the liner through flexibility enhancing cuts through a wall of the hypotube; and

FIG. 4 is an enlarged cross-sectional representation of a distal portion of the catheter of FIG. 1, showing bonding of a distal portion of the polymer layer, which extends beyond a distal end of the hypotube, to a distal portion of the liner, which also extends beyond the distal end of the hypotube.

### DETAILED DESCRIPTION

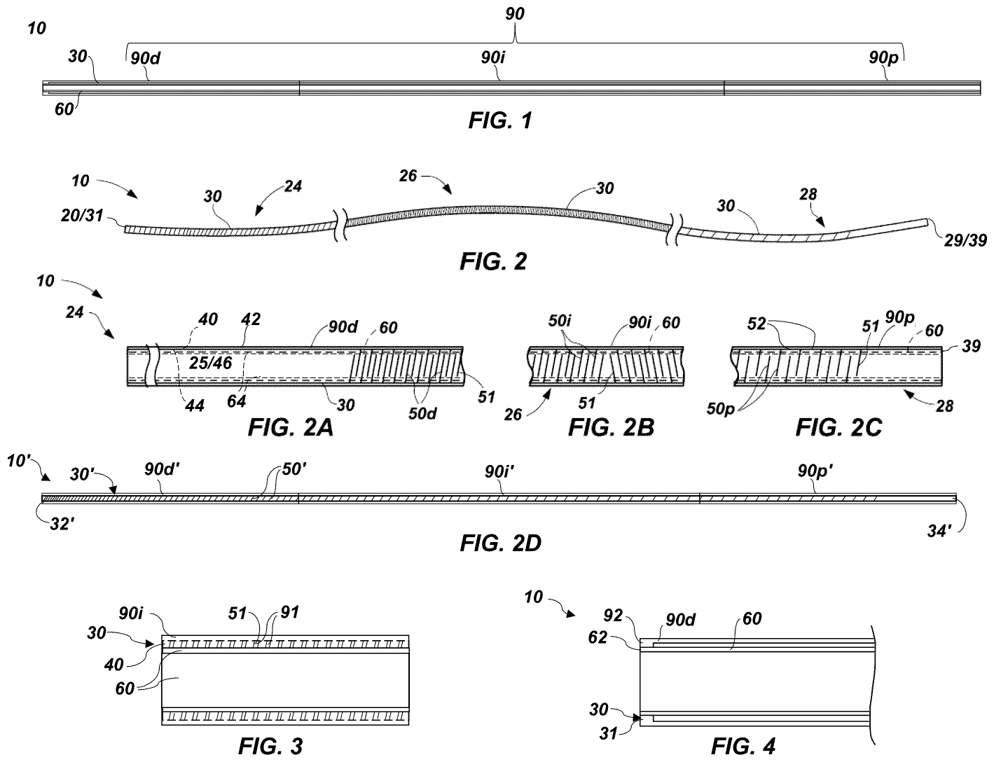

FIGS. 1, 3, and 4 illustrate an embodiment of a catheter 10 that includes a hypotube 30, an optional liner 60 within the hypotube 30, and a polymer layer 90 over the hypotube 30. The hypotube 30 extends along substantially an entire length of the catheter 10. The optional liner 60 defines an interior of the length of the catheter 10. The polymer layer 90 defines an exterior of the length of the catheter 10 and comprises a plurality of regions 90d, 90i, and 90p with flexibilities that differ, or vary, from one another.

As shown in FIG. 2, along its length, the catheter 10 includes a distal end 20, a distal portion 24, an intermediate portion 26, a proximal portion 28, and a proximal end 29. A lumen 25 (FIG. 2A) extends through the catheter 10. The distal end 20 of the catheter 10 is capable of being introduced into a body of a subject and advanced through the body of the subject (e.g., along a pathway, such as the subject's blood vessels, or vasculature, other vessels, or other tubes or passages) to a target site, where a procedure is to be performed. Upon advancement of the distal end 20 to the target site, the procedure that is to be performed may be performed through the lumen 25 and the distal end 20 of the catheter 10.

The intermediate portion 26 of the catheter 10, which may comprise a majority of the length of the catheter 10, is configured to enable advancement of the distal end 20 of the catheter 10 through the subject's body to the target site, to reside within the body of the subject once the distal end 20 has been advanced to the target site, to enable further movement of distal end 20 relative to the target site, and to enable removal of anything carried by the catheter 10 (e.g., tissue samples, debris, devices, etc.) and removal of the catheter 10 itself from the body of the subject. During advancement and removal of the catheter 10, a healthcare professional may hold the intermediate portion 26 at one or more locations to respectively push the catheter 10 into the subject's body and pull the catheter 10 out of the subject's body.

The proximal portion 28 of the catheter 10 is configured to reside outside of the subject's body during advancement of the catheter, use of the catheter 10, and removal of the catheter 10 from the subject's body. A proximal end 29 of the proximal portion 28 of the catheter 10 may have a configuration that enables it to couple to one or more devices that are to be used externally by a healthcare professional to perform one or more procedures at a target site (or a plurality of target sites) within the subject's body.

Turning now to FIGS. 2, 2A, 2B, and 2C, the hypotube 30 may comprise an elongated tube defined by a wall 40 with an outer surface 42 and an inner surface 44. The inner surface 44 of the wall 40 defines a lumen 46 that extends through a length of the hypotube 30. The hypotube 30 may be formed from any of a variety of suitable materials. The material from which the hypotube 30 is formed may be rigid, but have some flexibility when used to form extremely thin structures, such as the wall 40 of the hypotube 30. Examples of such materials include stainless steel (e.g., 304 stainless steel, 316 stainless steel, 316L stainless steel, etc.), metal alloys (e.g., nickel chromium (NiCr) steel, nitinol, cobalt/chromium, etc.), and various plastic materials. The dimensions of the hypotube 30 (e.g., its outer diameter, the thickness of its wall 40, its inner diameter, its length, etc.) may be suitable for one or more desired uses of the catheter 10. As examples, the outer diameter of the hypotube 30 may be about 0.005 inch (about 125 μm) to about 0.085 inch (about 2.2 mm, or 6.4 F) or larger (e.g., 7 F, 8 F, etc.). The inner diameter of the hypotube 30, or the diameter of the lumen 46 of the hypotube 30, may be about 0.002 inch (about 50 μm) to about 0.079 inch (about 2 mm) or larger. Since a hypotube 30 is used to define the catheter 10, even microcatheter embodiments of the catheter 10 may have outer diameters, wall thicknesses, and inner diameters that are uniform or substantially uniform (i.e., within acceptable tolerances) along their entire lengths. Such catheters 10 may have lengths of 150 cm or more than 150 cm (e.g., 175 cm, 200 cm, 220 cm, etc.) while providing desired or required levels of pushability, trackability, and torqueability.

Flexibility enhancing features 50d, 50i, and 50p of the hypotube 30 may be respectively located at the distal portion 24, the intermediate portion 26, and/or the proximal portion 28 of the catheter 10. The flexibility enhancing features 50d, 50i, and 50p may comprise flexibility enhancing cuts 51 that may extend through the wall 40 of the hypotube 30, from the outer surface 42 of the wall 40 to the inner surface 44 of the wall 40. Although the flexibility enhancing features 50d shown in FIG. 2A do not extend substantially or completely to a distal end of the hypotube 30, embodiments of catheters that include hypotubes 30 with cuts 51 and flexibility enhancing features 50d that extend substantially to or completely to the distal end of the hypotube are also within the scope of the disclosure.

The cuts 51 of a flexibility enhancing feature 50d, 50i, 50p may be helically oriented and/or circumferentially oriented. Cuts 51 that are helically oriented may be oriented at a non-perpendicular angle to longitudinal axis of the hypotube 30 when the hypotube 30 is straight. A cut 51 that is helically oriented may extend partially around the hypotube 30 (e.g., around at least about 25% of the hypotube, or have an arc length of at least about 0.5π, or 90°; around about 50% of the hypotube, or have an arc length of about π, or 180°; around up to 95% of the hypotube 30, or have an arc length of up to about 1.9π, or 342°, etc.) or completely around the hypotube 30 (i.e., have an arc length of 2π, or 360°, or greater), provided that a helically arranged series of cuts 51 does not facilitate elongation of the hypotube 30. The cuts 51 may comprise a continuous spiral that extends along at least a portion of the hypotube 30, such as an entirety of a cut portion of the hypotube 30, a distal end of a cut portion of the hypotube 30, etc. Cuts 51 that are circumferentially oriented may be oriented perpendicular to, or normal to, the longitudinal axis of the hypotube 30 when the hypotube 30 is straight. A circumferentially oriented cut 51 may extend partially around the hypotube 30 (e.g., up to about 95% of the distance around the hypotube 30, etc.).

Solid regions between ends of adjacent cuts 51 may define a spine 52 of a flexibility enhancing feature 50d, 50i, 50p. The spine 52 may be oriented helically or longitudinally. A flexibility enhancing cut 50d, 50i, 50p may include a single spine 52 or a plurality of spines 52 (e.g., two spines 52, three spines 52, four spines 52, etc.), depending upon the arc length each cut 51 that defines the flexibility enhancing feature 50d, 50i, 50p. As each spine 52 is defined at least in part by solid regions of the hypotube 30, each spine 52 may resist bending. The width(s) of the spine(s) 52, the number of spines 52, and the arrangement of spines 52 around the circumference of a flexibility enhancing feature 50d, 50i, 50p may at least partially dictate the column strength of the flexibility enhancing feature 50d, 50i, 50p, or the ability of the flexibility enhancing feature 50d, 50i, 50p to resist compression. The width of each spine 52 and the number of spines 52 around the circumference of the hypotube 30 may contribute to the flexibility/stiffness of the flexibility enhancing feature 50d, 50i, 50p. The orientation of each spine 52 on the flexibility enhancing feature 50d, 50i, 50p may determine the direction(s) in which the flexibility enhancing feature 50d, 50i, 50p may be deflected, or bend. Thus, a flexibility enhancing feature 50d, 50i, 50p may impart the catheter 10 with steerability without adding to the thickness, or outer diameter, of the catheter 10 and without decreasing the size (e.g., inner diameter, etc.) of the lumen 25 of the catheter 10.

The orientation of a cut 51 through the through the wall 40 of the hypotube 30 may be perpendicular to a tangent to the outer surface 42 of the wall 40 (i.e., it may extend the shortest possible distance through the wall 40, i.e., straight through the wall). Alternatively, a cut 51 that forms a flexibility enhancing feature 50d, 50i, 50p (each of which may also be referred to as a flexibility enhancing feature 50) may extend through the wall 40 at a non-perpendicular angle to a tangent to the outer surface 42 of the wall 40 (i.e., diagonally).

Each cut 51 may be performed by any of a variety of suitable processes, including, without limitation, by laser cutting techniques. In some embodiments, a laser beam with a nominal width, or kerf, of 0.0012 inch or less may be used. Smaller laser beam widths may be used to cut sharper features. Defocused laser beams with widths of up to about 0.0012 inch may be used to create angled cuts or shaped cuts 51 (e.g., hourglass shaped cuts, etc.), which may define smooth corners at the inner diameter of a catheter 10. The optional creation of cuts 51 with smooth corners at the inner diameter of a catheter 10 may facilitate the introduction of the liner 60 into the lumen 46, and prevent the cuts 51 from damaging the liner 60.

The flexibility enhancing features 50d, 50i, 50p may be arranged differently at different locations along the length of the catheter 10. As nonlimiting examples, the flexibility enhancing features 50d on a distal portion 24 of the catheter 10 may be positioned closer together (i.e., have a tighter pitch) than the flexibility enhancing features 50i of the intermediate portion 26 of the catheter 10 and the flexibility enhancing features 50p of the proximal portion 28 of the catheter 10. Such an arrangement may render the part of the distal portion 24 where the flexibility enhancing features 50d are located to be more flexible and, thus, to have greater trackability than the parts of the intermediate portion 26 and the proximal portion 28 where flexibility enhancing features 50i and 50p are respectively located. The flexibility enhancing features 50i and 50p of the intermediate portion 26 and the proximal portion 28 may render these portions of the catheter 10 more rigid than the distal portion 24 and, thus, impart these portions of the catheter 10 with more pushability than the distal portion 24. Additionally, the flexibility enhancing features 50p of the proximal portion 28 may be spaced further apart from one another than the flexibility enhancing features 50i of the intermediate portion 26, making the proximal portion 28 more rigid and, therefore, imparting the proximal portion 28 with greater pushability than the intermediate portion 26.

In addition to the spacing, or pitch, between adjacent flexibility enhancing features 50d, 50i, 50p, other factors, such as the lengths of the cuts 51, orientations of the cuts 51, and positioning (e.g., spacing, etc.) between series of circumferentially oriented or helically oriented flexibility enhancing features 50d, 50i, 50p may at least partially contribute to the flexibility and/or rigidity of a part of the hypotube 30. With respect to the lengths of the cuts 51, longer cuts 51 may create more flexibility in the hypotube 30 than shorter cuts 51.

Turning briefly to FIG. 2D, another embodiment of a catheter 10′ is schematically illustrated that includes an embodiment of a hypotube 30′ with cuts 50′ that enhance a flexibility of the hypotube 30′. The cuts 50′ have a pitch that gradually changes over a length of the hypotube 30′, which affects the spacing between adjacent cuts 51′ as well as the orientations of the cuts 51′ (e.g., cuts 51′ that are spaced farther apart from one another may also be offset at a greater angle from a circumference around the hypotube 30′ than cuts 51′ that are positioned more closely to each other). For example, the pitch of the cuts 50′ may increase from a more proximal location 34′ along the length of the hypotube 30′ to a more distal location 32′ along the length of the hypotube 30′. In an even more specific example, the increase in the pitch of the cuts 50′ may be constant. Proximal to the more proximal location 34′, the hypotube 30′ may include an uncut region 36′, which may be stiffer than any cut region of the hypotube 30′. The spacing between the cuts 50′ at the more proximal location 34′ may make the more proximal location 34′ more flexible than the proximally adjacent uncut region 36′, but the more proximal location 34′ may still be relatively stiff. The stiffness of the hypotube 30′ may decrease and, thus, a flexibility of the hypotube 30′ may increase, from the more proximal location 34′ to the more distal location 32′.

The catheter 10′ may also include a polymer layer 90′ with regions 90d′, 90i′, 90p′ that have flexibilities (e.g., hardnesses, thicknesses, etc.) that differ from each other. Although three regions 90d′, 90i′, 90p′ are depicted, the polymer layer 90′ may include fewer than three regions 90d′, 90i′, 90p′ (i.e., two regions) or more than three regions 90d′, 90i′, 90p′ (e.g., four (4) regions, five (5) regions, etc.). The flexibility of each region 90d′, 90i′, 90p′ of the polymer layer 90′ may further (i.e., in addition to flexibility enhancing cuts 51′) affect the flexibility of a corresponding region of the catheter 10′.

With reference to FIGS. 1-2D, the hypotube 30, 30′ may have any suitable dimensions. Without limitation, the wall 40 of the hypotube 30, 30′ may have a thickness (i.e., a distance along a radius of the hypotube 30) of about 0.0015 inch (about 38 μm) to about 0.006 inch (about 150 μm); the inner diameter of the hypotube 30, or the diameter of the lumen 46 of the hypotube 30, may be about 0.002 inch (about 50 μm) to about 0.085 inch (about 2.2 mm) or larger (e.g., 7 F, 8 F, etc.); and the outer diameter of the hypotube 30 may be about 0.005 inch (about 125 μm) to about 0.079 inch (about 2 mm) or larger.

The optional liner 60 lines the inner surface 44 of the wall 40 of the hypotube 30, 30′, or the lumen 46 of the hypotube 30, 30′. The liner 60 may extend along the entire length of the lumen 46 and of the hypotube 30, 30′ and line an entire inner surface 44 of the hypotube 30, 30′. Thus, the liner 60 may define the lumen 25 of the catheter 10 and an inner surface 64 of the liner 60 may define the inner diameter of the catheter 10. The liner 60 may have a thickness of about 0.00025 inch (about 6.35 μm) to about 0.00075 inch (about 19.1 μm) (e.g., about 0.0005 inch (about 12.7 μm), etc.). Optionally, the liner 60 may be longer than the lumen 46 and the hypotube 30. Such a liner 60 may extend beyond a distal end 31 and/or a proximal end 39 of the hypotube 30.

The inner surface 64 of the optional liner 60 may be smoother than, or have a lower coefficient of friction than, the inner surface 44 of the hypotube 30. Thus, the liner 60 may enhance the ability of fluids to flow through the lumen 25 of the catheter 10. In a specific embodiment, the liner 60 may comprise polytetrafluoroethylene (PTFE).

As best seen in FIGS. 1, 3, and 4, the polymer layer 90, 90′ (FIG. 2D) is located over the exterior surface 42 of the wall 40 of the hypotube 30, 30′ (FIG. 2D). The polymer layer 90, 90′ may extend along an entire length of the hypotube 30, 30′. Optionally, the polymer layer 90, 90′ may be longer than the hypotube 30, 30′, such that the polymer layer 90, 90′ may extend beyond the distal end 31 and/or the proximal end 39 of the hypotube 30, 30′.

The polymer layer 90, 90′ and each section 90d, 90i, 90p, 90d′, 90i′, 90p′ thereof may have a thickness of about 0.0010 inch (about 25.4 μm) to about 0.0025 inch (about 63.5 μm) and, thus, contribute about 0.002 inch (about 50.8 μm) to about 0.005 inch (about 127 μm) to the OD of the catheter 10, 10′. The thickness of one section 90d, 90i, 90p; 90d′, 90i′, 90p′ of the polymer layer 90, 90′ may differ from the thickness of at least one other section 90d, 90i, 90p; 90d′, 90i′, 90p′ of the polymer layer 90, 90′.

Each section 90d, 90i, 90p, 90d′, 90i′, 90p′ of the polymer layer 90, 90′ may comprise a polymer that may reflow when subjected to appropriate conditions (e.g., heat, pressure, etc.). For example, each section 90d, 90i, 90p; 90d′, 90i′, 90p′ of the polymer layer 90, 90′ may comprise a TPE, such as a polyether block amide (PEBA) (e.g., that available as PEBAX® polymer from Arkema SA of Colmbes, France; that available as VESTAMID® E from Evonik Industries AG of Essen, Germany, etc.) or a polyamide 12, or nylon 12 (e.g., that available as VESTAMID® Care ML21 polymer from Evonik Industries AG, etc.). The polymer from which each section 90d, 90i, 90p; 90d′, 90i′, 90p′ of the polymer layer 90, 90′ may differ (at least in hardness, or durometer) from at least one other section 90d, 90i, 90p; 90d′, 90i′, 90p′ of the polymer layer 90′. As an example, a proximal portion 90p, 90p′ of the polymer layer 90, 90′ may be harder than the intermediate portion 90i, 90′, which may in turn be harder than the distal portion 90d, 90d′. In an even more specific example, a hardness of the polymer layer 90, 90′ may transition from about 70 Shore D or more at its proximal portion 90p, 90p′ to about 25 Shore D or less at its distal portion 90d, 90d′, with one or more intermediate portions 90i, 90i′ having intermediate hardnesses (e.g., about 65 Shore D, about 60 Shore D, about 55 Shore D, about 50 Shore D, about 45 Shore D, about 40 Shore D, about 35 Shore D, about 30 Shore D, etc.).

As depicted by FIG. 3, portions 91 of the polymer layer 90 (and the embodiment of the polymer layer 90′ shown in FIG. 2D) may extend into and through cuts 51 in the wall 40 of the hypotube 30. These portions 91 may contact the liner 60 within the lumen 46 of the hypotube 30 and may be bonded to the liner 60. As illustrated by FIG. 4, a distal end 92 of the polymer layer 90 (and the embodiment of the polymer layer 90′ shown in FIG. 2D) may extend beyond the distal end 31 of the hypotube 30. In embodiments where the distal end 92 is superimposed by a distal end 62 of the liner that also extends beyond the distal end 31 of the hypotube 30, the distal end 92 of the polymer layer 90 may be bonded to the distal end 62 of the liner 60, providing a structure that many serve as an atraumatic tip of the catheter 10.

Optionally, the polymer layer 90, 90′ may also bond to one or more surfaces of the hypotube 30, 30′. For example, the polymer layer or the portions 91 thereof 90 may bond to the outer surface 42 of the wall 40 of the hypotube 30 (see FIG. 2A), surfaces that define the distal end 31 and/or proximal end 39 of the hypotube 30 (see FIGS. 4 and 2C, respectively), and/or surfaces that define the cuts 51 through the wall 40 of the hypotube 30 (see FIG. 3).

A catheter 10 of this disclosure may withstand pressures of at least 1,200 psi (about 8.275×10³ kpa) (i.e., the maximum pressure of a current pressure injectors used by medical professionals, (e.g., in radiology, such as in angiography procedures, etc.)).

The inclusion of the optional liner 60 within the lumen 46 of the hypotube 30 may provide a surface with constant lubricity along a length of the catheter 10. By using a hypotube 30 with a liner 60 in its lumen 46 to form a catheter 10, the catheter 10 may have a small outer diameter (e.g., 6 F or less), an inner diameter that is at least as large as the inner diameter of a comparably sized microcatheter that has been manufactured from a polymer while retaining lubricity along its length, and a length that exceeds the length of a comparably sized microcatheter that has been manufactured from a polymer while having desired or required levels of pushability, trackability, and torqueability (e.g., 220 cm, 175 cm, etc., for catheters that are smaller than 6 F).

While the above-described catheters 10 and 10′ include hypotubes 30 and 30′ with flexibility enhancing cuts 51 and 51′ that define flexibility enhancing features 50 and 50′ that impart different regions along the length of the hypotubes 30 and 30′ with different flexibilities, it should be noted that a catheter of this disclosure may also include a hypotube with flexibility enhancing cuts that impart the hypotube with a uniform flexibility along substantially its entire length. Any variation in the flexibility of different regions along the length of such a catheter may be imparted to the catheter by way of a polymer coating that has varied flexibility (e.g., hardness, thickness, etc.) along its length.

A method for manufacturing a catheter 10, 10′ of this disclosure may include selecting and providing a hypotube 30, 30′ of a desired material and dimensions (e.g., outer diameter, inner diameter, etc.). Flexibility enhancing features 50, 50′ may then be formed in or on the hypotube 30. In a specific embodiment, each flexibility enhancing feature 50, 50′ may be formed by defining flexibility enhancing cuts 51, 51′ in the hypotube 30, 30′. Each flexibility enhancing cut 51, 51′ may be made by any of a variety of suitable processes, including, without limitation, by laser cutting techniques.

Once the flexibility enhancing features 50, 50′ and any other features have been defined in the hypotube 30, 30′, a mandrel may be inserted into the lumen 46 (FIG. 2A) of the hypotube 30, 30′. The mandrel may carry an optional liner 60 on its outer surface. As the mandrel is introduced into the lumen 46, its outer surface may be positioned adjacent to or against an inner surface 44 of a wall 40 (FIG. 2A) of the hypotube 30, 30′. In embodiments where a mandrel is used to introduce a liner 60 into the lumen 46, the mandrel may hold the liner 60 adjacent to or against the inner surface 44 of the wall 40 of the hypotube 30.

As a nonlimiting example, the optional liner 60 may comprise a preformed tube with outer cross-sectional dimensions, taken normal to a longitudinal axis of the liner 60, that enable the liner 60 to be received by the inner diameter of the lumen 46 of the hypotube 30. Such a liner 60 may comprise a suitable polymer (e.g., PTFE, etc.).

A polymer layer 90, 90′ may be applied to an exterior surface 42 of the wall 40 (FIG. 2A) of the hypotube 30, 30′. Such a polymer layer 90, 90′ may comprise a series of preformed tubes, each of which is referred to as a section 90d, 90i, 90p, 90d′, 90i′, 90p′ of the polymer layer 90, 90′, positioned adjacent to one another over the length of the hypotube 30, 30′. Each section 90d, 90i, 90p, 90d′, 90i′, 90p′ may be formed from a suitable polymer. A lumen through the preformed tube of each section 90d, 90i, 90p, 90d′, 90i′, 90p′ of the polymer layer 90, 90′ may have cross-sectional dimensions, taken normal to a longitudinal axis of the preformed tube, that enable the preformed tube to receive the OD of the hypotube 30, 30′. Each performed tube may be cut to a desired length, and the hypotube 30, 30′ may be introduced into the lumens of the preformed tubes. A polymer of each preformed tube may reflow when it is subjected to appropriate conditions (e.g., heat, pressure, etc.). For example, each section 90d, 90i, 90p, 90d′, 90i′, 90p′ of the polymer layer 90, 90′ may comprise a TPE, such as a PEBA, a polyamide 12, or the like.

A heat shrink tube (not shown) may be placed over the polymer layer 90, 90′. The heat shrink tube may be placed over the polymer layer 90, 90′ once the sections 90d, 90i, 90p, 90d′, 90i′, 90p′ of the polymer layer 90, 90′ are in place over the hypotube 30, 30′. The heat shrink tube may completely cover the polymer layer 90, 90′. The heat shrink tube may be formed from a contractible, or shrinkable, material (e.g., a heat shrinkable material, such as a polyolefin (e.g., PET; etc.), etc.).

Once the polymer layer 90, 90′ is in place over the hypotube 30, 30′ and the heat shrink tube is in place over the polymer layer 90, 90′, the polymer layer 90, 90′ and the heat shrink tube may be subjected to conditions (e.g., heat, pressure, etc.) that will cause the polymer from which each section 90d, 90i, 90p, 90d′, 90i′, 90p′ of the polymer layer 90, 90′ is formed to reflow. As the polymer of each section 90d, 90i, 90p, 90d′, 90i′, 90p′ of the polymer layer 90, 90′ reflows, adjacent sections 90d, 90i, 90p, 90d′, 90i′, 90p′ may flow together and, thus, become continuous with each other. The conditions to which the polymer layer 90, 90′ and the heat shrink tube are subjected may also cause the heat shrink tube to shrink to dimensions that will force the polymer(s) of the polymer layer 90, 90′ against the outer surface 42 of the wall 40 (FIG. 2A) of the hypotube 30, 30′, into cuts 51, 51′ through the wall 40 of the hypotube 30, 30′, and against any liner 60 within the hypotube 30, 30′. Heating and shrinkage of the heat shrink may also mold the polymer of the polymer layer 90, 90′ to desired dimensions (e.g., a desired OD, etc.). The polymer layer 90, 90′ and the heat shrink may be heated to a temperature of at least 125° C., a temperature of at least 133° C., a temperature of about 160° C. to about 200° C., or the like to cause the polymer of the polymer layer 90 to reflow and the heat shrink tube to contract.

In addition to causing the polymer(s) of the polymer layer 90, 90′ to reflow and the heat shrink tube to contract, the conditions to which the polymer layer 90, 90′ is subjected may enable the polymer(s) of the polymer layer 90, 90′ to bond to portions of any liner 60 the polymer(s) contact(s). For example, with reference to FIG. 3, the portions 91 of the polymer layer 90 that extend through cuts 51 through the wall 40 of the hypotube 30 may be bonded to portions of any liner 60 that are exposed to the cuts 51. As another example, with reference to FIG. 4, a distal portion 92 of the polymer layer 90 that extends beyond, or overhangs, the distal end 31 of the hypotube 30 may bond to a distal portion 62 of any liner 60 that also extends beyond the distal end 31 of the hypotube. Together, these bonded distal portions 62 and 92 of the liner 60 and polymer layer 90, respectively, may define a tip of the catheter 10. In embodiments where a mandrel is used to introduce a liner 60 into the lumen 46 of the hypotube 30 and hold the liner 60 against an inner surface 44 of the wall 40 of the hypotube 30, the mandrel may maintain an ID of the lumen 25 of the catheter 10 through the distal tip as the distal portion 92 of the polymer layer 90 bonds to the distal portion 62 of the liner 60 to provide an atraumatic tip at the distal end 20 of the catheter 10.

As the polymer(s) of the polymer layer 90, 90′ is (are) subjected to conditions that cause the polymer(s) to reflow and that shape the polymer layer 90, 90′, the polymer(s) of the polymer layer 90, 90′ may also bond to surfaces of the hypotube 30, 30′ that the polymer(s) contact(s). Examples of such surfaces include the outer surface 42 of the wall 40 of the hypotube 30, 30′ (see FIG. 2A), surfaces of the cuts 51, 51′ that extend through the wall 40 (see FIG. 3), and surfaces that define the distal end 31 and proximal end 39 of the hypotube 30 (see FIGS. 4 and 2C, respectively).

Although the disclosure provides many specifics, the specifics should not be construed as limiting the scope of any of the claims, but merely as providing illustrations of some embodiments of elements and features of the disclosed subject matter that fall within the scopes of the claims. Other embodiments of the disclosed subject matter may be devised that are also within the scopes of the claims. The scope of each claim is limited only by its plain language and the legal equivalents thereto.

What is claimed:

1. A catheter, comprising:
a hypotube including a wall with an inner surface and an outer surface and flexibility-enhancing cuts extending through the wall, from the inner surface to the outer surface; and
a polymer layer on the outer surface of the wall of the hypotube and comprising a plurality of sections having different flexibilities.

2. The catheter of claim 1, wherein the plurality of sections of the polymer layer have different hardnesses from each other.

3. The catheter of claim 1, wherein the polymer layer includes:
a distal section having a distal flexibility; and
a proximal section having a proximal flexibility, the distal flexibility being more flexible than the proximal flexibility.

4. The catheter of claim 3, wherein the polymer layer further includes:
at least one intermediate section having at least one intermediate flexibility, the at least one intermediate flexibility being more flexible than the proximal flexibility of the proximal section and less flexible than the distal flexibility of the distal section.

5. The catheter of claim 1, wherein ends of adjacent sections of the plurality of sections of the polymer layer are continuous with each other.

6. The catheter of claim 5, wherein ends end of the adjacent sections of the plurality of sections of the polymer layer comprise transitions between the adjacent sections.

7. The catheter of claim 1, further comprising:
a liner on the inner surface of the wall of the hypotube.

8. The catheter of claim 7, wherein the liner is bonded to portions of the polymer layer that extend through the flexibility-enhancing cuts of the hypotube.

9. A catheter, comprising:
a hypotube including a wall with an inner surface and an outer surface and flexibility enhancing cuts extending through the wall, from the inner surface to the outer surface; and
a polymer layer on the outer surface of the wall of the hypotube and comprising:
a proximal section over a proximal portion of the hypotube, having a proximal hardness, and imparting the proximal portion of the hypotube with a proximal flexibility;
at least one intermediate section over at least one intermediate portion of the hypotube, having at least one intermediate hardness, and imparting the at least one intermediate portion of the hypotube with at least one intermediate flexibility, the at least one intermediate hardness being softer than the proximal hardness, the at least one intermediate flexibility being more flexible than the proximal flexibility; and
a distal section over a distal portion of the hypotube, having a distal hardness, and imparting the distal portion of the hypotube with a distal flexibility, the distal hardness being softer than the at least one intermediate hardness, the distal flexibility being more flexible than the at least one intermediate flexibility.

10. The catheter of claim 9, wherein the hypotube comprises stainless steel.

11. The catheter of claim 9, wherein the hypotube comprises a shape memory alloy.

12. The catheter of claim 9, wherein the polymer layer comprises a polyether block amide (PEBA).

13. A method for manufacturing a catheter, comprising:
cutting a hypotube to define flexibility enhancing cuts in the hypotube;
introducing a plurality of sections of a polymer layer over an outer surface of the hypotube, the plurality of sections being sequentially arranged over a length of the hypotube;
providing a heat shrink over the polymer layer; and
heating the heat shrink and the polymer layer to a temperature sufficient to shrink the heat shrink and cause a material of the polymer layer to flow, securing adjacent ends of adjacent sections of the plurality of sections of the polymer layer to each other.

14. The method of claim 13, wherein cutting the hypotube comprises cutting the hypotube to define the flexibility enhancing cuts in a spiral arrangement.

15. The method of claim 13, wherein introducing the plurality of sections over the outer surface of the hypotube comprises introducing a plurality of sections having different flexibilities over the outer surface of the hypotube.

16. The method of claim 15, wherein introducing the plurality of sections over the outer surface of the hypotube comprises introducing a plurality of sections having different hardnesses over the outer surface of the hypotube.

17. The method of claim 16, wherein introducing the plurality of sections of the polymer layer over the outer surface of the hypotube comprises:
introducing a distal section of the polymer layer with a distal hardness over a distal portion of the hypotube; and
introducing a proximal section of the polymer layer with a proximal hardness over a proximal portion of the hypotube, the distal hardness being softer than the proximal hardness.

18. The method of claim 17, wherein introducing the plurality of sections of the polymer layer over the outer surface of the hypotube further comprises:
introducing at least one intermediate section of the polymer layer with at least one intermediate hardness over at least one intermediate portion of the hypotube, the at least one intermediate hardness being softer than the proximal hardness of the proximal section of the polymer layer but harder than the distal hardness of the distal section of the polymer layer.

19. The method of claim 13, wherein heating the heat shrink and the polymer layer comprises reflowing the ends of the adjacent sections of the polymer layer into each other to form a transition between the adjacent sections.

20. The method of claim 13, wherein heating the heat shrink and the polymer layer enables material of the polymer layer to flow into the flexibility-enhancing cuts in the hypotube.

21. The method of claim 13, further comprising:
introducing a liner into a lumen of the hypotube, against an inner surface of a wall of the hypotube.

22. The method of claim 21, wherein heating the heat shrink and the polymer layer enables the material of the polymer layer to flow into the flexibility enhancing cuts of the hypotube.

23. The method of claim 22, wherein heating the heat shrink and the polymer layer further enables the material of the polymer layer to bond to the liner.
